(19) United States
(12) Patent Application Publication
(10) Pub. No.: US 2002/0034485 A1
(43) Pub. Date: Mar. 21, 2002

(54) Hair tonic for prevention or treatment of hair loss

(76) Inventors: Friedrich Noser, Bonnefontaine (CH); Ursula Hehner, Brensbach (DE); Bernd Wagenknecht, Mainz (DE)

Correspondence Address: STRIKER, STRIKER & STENBY, 103 East Neck Road, Huntington, NY, 11743, US

(21) Appl. No.: 09/909,289

(22) Filed: Jul. 19, 2001

(30) Foreign Application Priority Data
Jul. 22, 2000 (DE) 100 35 735.0

(51) Int. Cl. A61K 7/06

(52) U.S. Cl. 424/70.1

(57) ABSTRACT

The hair tonic for preventing or at least reducing hair loss includes at least one saturated or unsaturated C₁₀- to C₁₈-fatty acid, or a salt thereof, biotin and/or caffeine. The method of preventing or at least reducing hair loss includes applying the hair tonic to the affected area on the scalp or to an area on the head in which prevention of hair loss is desired.

### BACKGROUND OF THE INVENTION

The subject matter of the present invention is a cosmetic composition, especially a hair tonic, for preventing or treating hair loss containing an effective ingredient combination of fatty acids, biotin and/or caffeine. The subject matter of the present invention also includes a cosmetic method for increasing natural hair growth and for reduction of hair loss.

The growth of human hair occurs in a known manner according to a natural growth rhythm including a growth phase (anagen), a transition phase (catagen) and a resting phase (telogen). A normal natural hair cycle begins when a hair bottom end is ejected or cast away at the beginning of the anagen phase. Consequently from 80 to 100 hairs fall out daily from the scalp. This number is an approximate value, which can occur over a shorter time and also can be greater or smaller, without causing fear regarding natural hair growth. However when the amount of hair falling out daily exceeds this approximate normal amount, an irreversible or reversible hair loss is occurring. The anagen follicle is very susceptible toward disturbing influences because of its nature as a highly reactive organ, whereas the telogen follicle scarcely reacts at all. The natural first reaction of the hair follicle is thus to rapidly switch into the telogen phase and to begin a fresh anagen phase as soon as the disturbing influence stops. Great disturbing influences lead from the loss of hair thickness to the anagen hair loss, which occurs when the hair follicle has no more time remaining for change into the telogen phase. No strict relationship exists between the reaction of the hair organs and the type of disturbing influence.

Reversible hair loss is indeed widespread. For example, hair loss occurs after feverish and consumptive illnesses, as a result of an unbalanced diet, because of dietary iron deficiency, due to albumin-free diet, during cures involving fasting and as a result of taking medicine to treat cancer. Hair loss also occurs in women after a birth. Gestagen, the hormone, which is formed during the pregnancy in a great amount, synchronizes the growth cycle of the hair on the head. A very high ratio of anagen hair to telogen hair is the result. After the birth this ratio changes in the opposite manner, which means many anagen hairs change into telogen hairs. After several months a shift into the anagen phase occurs during which the hairs can fall out simultaneously leading to a conspicuous bare spot in the hair on the head. When the new anagen hairs have reached their original length, the original state is again reached. Hair loss after massive psychic stress is another form of reversible hair loss. How this type of hair loss occurs is unknown. It appears that stress, which can impair many functions of our metabolism, also affects the operation of the hair organ running continuously at maximum performance.

Androgenic hair loss, and/or the conversion of terminal hair into vellus hair, is the classical form of hair loss that has been classified up to now as largely irreversible. It may start in early manhood (at around 17) and may be complete by about age 50. The two most important biologically active forms of the androgens are testosterone and 5-alpha-dihydrotestosterone derived from it. An enzyme, 5-alpha-reductase, converts testosterone into dihydrotestosterone, which is the active hormone form for influencing the growth of hair. Androgens, which act on the hair and body hair, are soloists in the hormone orchestra. It is noteworthy that they act in an exact opposite manner on the body hair than on the head hair. When they change terminal hairs on the scalp into scarcely visible vellus hair, they cause the vellus hair to grow in the beard region to produce signs of manhood in youths advancing into puberty. In older men hair loss due to old age, which predominantly involves follicle transformations of terminal follicles into intermediary follicles and vellus follicles, in both cases leads to formation of a further diffuse bare region in the still existing hair. In this stage of old age the hair requires special completely effective and specially suited care. Disturbances, which lead to a destruction of the hair organ, cause permanent baldness.

### SUMMARY OF THE INVENTION

It is an object of the present invention to provide a composition and a method that induce or guarantee healthful hair growth and formation of hair of natural beauty.

It is another object of the present invention to provide a composition and method that counteract hair changes and hair loss due to various causes, i.e. to prevent or at least significantly reduce them, to provide recovery from reversible hair loss, and to stop and ideally to reverse as much as possible the transformations that lead to irreversible, especially adrogenically caused, hair loss.

EP 0 116 439 describes hair tonics containing certain fatty acids as effective ingredients. Applications of these tonics include prevention of flaking, prevention of scalp urticaria and acceleration of hair growth, which was tested with shaved or clipped rabbits. This reference does not disclose that the hair tonics described in it reduce or prevent hair loss in men.

It has now been found that compositions containing a combination of fatty acids, biotin and/or caffeine are even more effective in promotion of hair growth, preventing or avoiding hair loss than individual ingredients that act on different sites essential for hair growth.

According to the invention a cosmetic composition, especially a hair tonic, for preventing or at least reducing hair loss, comprises

(A) at least one saturated or unsaturated C₁₀- to C₁₈-fatty acid or its salt, and

(B) at least one effective ingredient selected from the group consisting of biotin and caffeine.

A method of preventing or treating hair loss using a combination of the above-mentioned ingredients (A) and (B) is also part of the subject matter according to the invention.

The subject matter of the invention also includes a cosmetic method for augmenting natural hair growth and/or reducing hair loss, comprising the steps of:

a) inhibiting the action of 5-alpha-dihydrotestosterone on head hair thickness or abundance;

b) promoting keratinization of trichocytene by introduction of regulators, and

c) increasing blood supply in the region around the hair organ.

These steps are preferably performed one after the other or simultaneously by application of a single effective ingredient complex to the scalp. The method can be successfully performed by external application to the scalp of an effective ingredient complex or a composition that contains an effective ingredient complex in a physiologically compatible base. The effective ingredient complex comprises:

(A) a C₁₀- to C₁₈-fatty acid, preferably a C₁₂- to C₁₄-fatty acid, especially preferably lauric acid,

(B) biotin (vitamin H),

(C) caffeine (1,3,7-trimethylxanthine), as well as additional effective ingredients, which act on the site essential for hair growth, especially additional blood-supply promoting agents, tocopherols and their esters, nicotinic acid and its esters, nicotinamide, camphor, Royal jelly or blood supply promoting plant extracts.

When the effective ingredient complex is applied, the activity of the enzyme 5-alpha-reductase is reduced or halted so that less 5-alpha-dihydro-testosterone required for human hair loss is formed. Also the blood supply in the area of application is increased, as well as the flow of blood, and thus the nutrient supply in the hair organ. The keratinization, the tear resistance, the strength of the hair and thus the resistance of the hair to environmental influences and the treatments are increased. The anchoring of the hair bottom end in the scalp is strengthened. The number of lost hairs decreases and is stabilized at a minimum level.

C₁₀- to C₁₈-FATTY ACIDS

The fatty acid is preferably contained in an amount of from 0.01 to 10 percent by weight, especially preferably from 0.1 to 2 percent by weight. Suitable fatty acids include saturated or unsaturated, branched or unbranched fatty acids. Suitable fatty acids in particular include, for example, capric acid, lauric acid, myristic acid, isomyristic acid, palmitic acid, palmitoleic acid, stearic acid, oleic acid, linoleic acid, linolenic acid, parinaric acid and petroselinic acid or their cosmetically acceptable salts. The C₁₂- to C₁₄-fatty acids are particularly preferred, especially lauric acid and myristic acid or their combinations. Cosmetically acceptable salts include alkali metal, alkaline earth metal or ammonium salts. Fatty acids in the free form that has a higher activity, which are not salts, are especially preferred.

Biotin

Biotin is present in an amount of from 0.001 to 1 percent by weight, especially preferably from 0.005 to 0.1 percent by weight, in the composition according to the invention. Biotin belongs to the vitamin-B-complex (as B₇) and is also known as vitamin H. The chemical name for it is D-cis-hexahydro-2-oxothieno-[3,4-d]-imidazol-4-valeric acid. Use of biotin influences the quality of the hair positively. Biotin is the vitamin of keratinization, i.e. an improved supply of biotin to the hair organ improves keratinization. Biotin particularly increases the strength and tear resistance and generally the resistance of hair to outside or environmental influences and stressful treatments in the case of fine hair but also significantly for normal hair. Biotin strengthens the anchoring of the bottom of the hair in the scalp. As a result there is a decrease in the number of hairs falling out and the number lost is stabilized at a minimum value.

Caffeine

Caffeine is present in an amount of from 0.01 to 10 percent by weight, especially preferably from 0.1 to 2 percent by weight, in the compositions according to the invention. Caffeine is a plant alkaloid numbered among the purines and is also designated as theine, methyltheobromine or guaranine. The chemical name is 1,3,7-trimethyl xanthine. It is a material, which is found in a number of plants, especially in coffee beans, black tea, Brazil tea, cocoa hearts and cola nuts. Caffeine is vascodilating, i.e. increases the supply of blood to the hair organ, thus increases its nutrient supply and prolongs the hair growth cycle.

Hair Tonic

The hair tonic according to the invention can have a conventional base for a cosmetic composition, for example an aqueous base, an alcoholic base or an aqueous-alcoholic base with preferably at least 10 percent by weight water and at least 30 percent by weight alcohol. The hair tonic preferably contains from 70 to 99.5 percent by weight, especially preferably 90 to 99 percent by weight, of a solvent system. The hair tonic can contain lower univalent alcohols suitable for cosmetic purposes, such as ethanol and isopropanol, or multivalent alcohols, such as glycerol, ethylene glycol or propylene glycol. The composition according to the invention preferably has a pH value at which the fatty acid present is in the form of a free acid, especially a pH value in a range of from 3 to 7, especially preferably from 4 to 6.5, and most preferably from 4.5 to 6.

In an especially preferred embodiment the hair tonic contains both biotin and caffeine as well a C₁₂- to C₂₀-fatty acid. It has been found that the effectiveness of this effective ingredient complex is especially powerful because of the presence of all three different ingredients. This preferred hair tonic has the power to help form healthy hair, to counteract hair changes and hair loss, to promote recovery from reversible hair loss and to slow or stop irreversible hair slow.

Additional Effective Ingredients

An additional increase in the effectiveness of the compositions according to the invention is obtained by including additional effective ingredients, which act on the same or different sites essential for hair growth, for example additional vasodilating substances or substances that have blood supply stabilizing or promoting properties. In a particularly preferred embodiment the hair tonic according to the invention has at least one additional effective ingredient, which acts on at least one of the sites essential for hair growth, especially an additional vasodilating substance. A vasodilating substance in the sense of the invention is a substance, which increases the blood flow to the scalp after application and because of that especially increases the nutrient supply to the hair organ.

The additional effective ingredients are preferably contained in an amount of from 0.001 to 2, especially preferably from 0.01 to 1, percent by weight. The different tocopherols (alpha-, beta-,gamma- or delta-tocopherol), preferably alpha-tocopherol, and its esters, especially vitamin E (tocopherol acetate), tocopheryl succinate, tocopherylnicotinate or tocopherylpoly(oxyethylene)-succinate. Nicotinic acid and its derivatives, especially nicotinamde, methylnicotinate or tocopherylnicotinate and camphor, Royal jelly and vasociliating plant extracts, such as burned or distilled nettle extract (Urtica Diocia), roast chestnut extract (Aesculus Hippocastanum), Arnica extract (Arnica Montana), hay flower extract, fir-needle extract (pine) or Swedtia Chirata.

The hair tonic according to the invention can contain additional conventional additive ingredients that are commonly used in cosmetic compositions, such as perfume oils, in an amount of from 0.01 to 0.5 percent by weight; turbidity-inducing agents, such as ethylene glycol distearate, in an amount of about 0.2 to 5 percent by weight; surfactants, especially emulsifiers; solvating agents; preservatives, such as e.g. para-hydroxybenzoic acid, in an amount of from 0.01 to 1 percent by weight; buffer substances, such as sodium citrate or sodium phosphate, in an amount of from 0.1 to 1 percent by weight; care materials, such as hair-care-providing or skin-care-providing plant or vegetable extracts, protein hydrolyzates and silk hydrolyzates, lanolin derivatives, in an amount of from 0.1 to 5 percent by weight; physiologically compatible silicone derivative compounds, such as volatile or non-volatile silicone oils or high molecular weight siloxane polymers in an amount of from 0.05 to 20 percent by weight; light protecting agents, antioxidants, radical-trapping agents, anti-flaking agents, in an amount of about 0.01 to 2 percent by weight; hair luster-imparting agents, vitamins, combability improving agents and defatting agents.

The hair tonic according to the invention is employed according to a method in which the hair tonic is applied to the scalp in an amount sufficient to obtain the desired effect and is gently worked in.

The following examples should illustrate the invention claimed below in greater detail, but the details of these examples should not be considered as further limiting the claims appended hereinbelow.

### EXAMPLES

### Example 1

Hair Tonic

| | | |
|---|---|---|
| Caffeine | 1.0 | g |
| Lauric acid | 0.2 | g |
| Biotin | 0.01 | g |
| Panthenol | 0.6 | g |
| Menthol | 0.25 | g |
| Perfume | 0.2 | g |
| PEG-40 hydrogenated | 0.18 | g |
| castor oil | | |
| Tocopheryl acetate | 0.05 | g |
| Water | 45.0 | g |
| Ethanol | To 100 | g |

### Example 2

Hair Tonic

| | | |
|---|---|---|
| Caffeine | 0.5 | g |
| Myristic acid | 0.4 | g |
| Biotin | 0.01 | g |
| Panthenol | 0.6 | g |
| Menthol | 0.25 | g |
| Perfume | 0.2 | g |
| PEG-40 hydrogenated | 0.18 | g |
| castor oil | | |
| Tocopheryl acetate | 0.05 | g |
| Water | 45.0 | g |
| Ethanol | To 100 | g |

### Example 3

Hair Tonic

| | | |
|---|---|---|
| Caffeine | 1.0 | g |
| Lauric acid | 0.1 | g |
| Myristic acid | 0.1 | g |
| Biotin | 0.01 | g |
| Panthenol | 0.3 | g |
| Menthol | 0.1 | g |
| Perfume | 0.2 | g |
| PEG-40 hydrogenated | 0.18 | g |
| castor oil | | |
| Tocopheryl acetate | 0.05 | g |
| Water | 45.0 | g |
| Ethanol | To 100 | g |

### Example 4

Effectiveness Study

The effectiveness of the composition according to the invention was tested in a clinical study. A group of 18 male subjects (Group A(M)) and a group of 19 female subjects (Group A(W)) were each treated with an effective ingredient complex-containing product (4A(M)) and/or 4A(W)) and a group of 17 male subjects (Group B(M)) and a group of 18 female subjects (Group B(W)) were treated with a placebo (4B(M)) and/or 4B(W)). All the subjects had androgenic alopecia II and III, graded on the Hamilton scale.

The application occurred in all test subjects for a total of 90 days. During the first 45 days the preparation was applied daily. During the following 45 days it was applied twice weekly.

Effective-ingredient Containing Product 4A(M):

| | | |
|---|---|---|
| Lauric acid | 0.2 | g |
| Caffeine | 1.0 | g |
| Biotin | 0 01 | g |
| Panthenol | 0.6 | g |
| Tocopheryl acetate | 0.05 | g |
| Plant extract* | 0 1 | g |
| Menthol | 0.25 | g |
| PEG-40 hydrogenated castor oil | 0.18 | g |
| PEG-35 castor oil | 0.46 | g |
| Perfume | 0.5 | g |
| Water | To 100 | g |
| Ethanol, 95% | 55 | g |

*Extrapon ® 5-UK new (Dragoco): water, propylene glycol, ethoxydiglycol, Betula Alba, Equisetum urtica, Salvia Officinalis, Dioica Arvense, Rosmarinos Officinalis, Achillea Millefollium, inositol, calcium pantothenate, sodium benzoate, potassium sorbate.

* Extrapon® 5-UK new (Dragoco): water, propylene glycol, ethoxydiglycol, Betula Alba, Equisetum urtica, Salvia Officinalis, Dioica Arvense, Rosmarinos Officinalis, Achillea Millefollium, inositol, calcium pantothenate, sodium benzoate, potassium sorbate.

Effective-ingredient Containing Product 4A(W):

| | | |
|---|---|---|
| Lauric acid | 0.2 | g |
| Caffeine | 1.0 | g |
| Biotin | 0.01 | g |
| Panthenol | 0.6 | g |
| Tocopheryl acetate | 0.05 | g |
| Multivitamin-vegetable complex: | 0 1 | g |
| Soluvit ® Richter | | |
| Menthol | 0.25 | g |
| PEG-40 hydrogenated castor oil | 0.18 | g |
| PEG-35 castor oil | 0.46 | g |
| Perfume | 0.2 | g |
| Water | To 100 | g |
| Ethanol, 95% | 55 | g |

Comparison Product 4B(M):

| | | |
|---|---|---|
| Perfume | 0.5 | g |
| Ethanol, 95% | 55 | g |
| Water | To 100 | |

Comparison Product 4B(W):

| | | |
|---|---|---|
| Perfume | 0.2 | g |
| Ethanol, 95% | 55 | g |
| Water | To 100 | |

Tests to determine the amount of hair loss were performed at the beginning of treatment (t0), at 45 days (t45) and after 90 days (t90). The tests were performed according to three methods: an objective judgement in which the tests were performed and evaluated scientifically; a subjective judgement by the respective test subjects themselves and a quantitative standard measurement (pull test).

The subjective and the objective judgement results were graded according to a scale of 0 to 6:

0: no hair loss

2: slight hair loss

4: average/moderate hair loss

6: strong hair loss.

The quantitative standard measurement occurred according to the so-called pull-test, described in Cosmesi Dermatologica, N. 65, April-June 1998, p. 139. A hair strand of about 60 hairs is taken or grasped between the thumb and the index finger in the crown region about 2 to 3 cm above the outer ear. A constant traction or pull is exerted on it until at the distal part of the hair. Then the number of torn out hairs is counted. The pull test measures the number of loosely anchored hairs in the scalp. The hair condition or state (telogen or anagen) is then determined by microscopic examination of the hair root. The test is indicative for Telogen effluvium, Anagen effluvium, breakage of the hair root shaft, androgenic hair loss, localized and diffuse hair loss.

TABLE I
EFFECTIVENESS RESULTS FOR HAIR TREATMENT WITH THE
COMPOSITIONS ACCORDING TO THE INVENTION

| | | A(W) | A(M) | B(W) | B(M) |
|---|---|---|---|---|---|
| HAIR LOSS | t0 | 3.4 | 3.6 | 3.2 | 3.6 |
| (Objective) | t45 | 3.0 | 3.3 | 3.2 | 3.6 |
| | t90 | 2.4 | 2.7 | 3.1 | 3.4 |
| | Δ⁽¹⁾ | 73.7% | 72.2% | 11.1% | 17.6% |
| HAIR LOSS | t0 | 3.4 | 3.8 | 3.1 | 3.4 |
| (Subjective) | t45 | 2.8 | 3.1 | 3.1 | 3.3 |
| | t90 | 2.3 | 2.6 | 2.9 | 3.1 |
| | Δ⁽¹⁾ | 78.9% | 77.8% | 16.7% | 23.5% |
| Pull Test | t0 | 10.9 | 11.7 | 10.0 | 11.5 |
| | t45 | 9.7 | 10.1 | 9.9 | 11.3 |
| | t90 | 6.8 | 7.2 | 9.4 | 10.2 |
| | Δ⁽²⁾ | 37.6% | 38.5% | 6.0% | 11.3% |

Δ⁽¹⁾ number of test subjects in %, in which hair loss was reduced.
Δ⁽²⁾ reduction of number of only loosely anchored hairs in the scalp, in %.

All results were significant according to statistical analysis. The results show a definite reduction of hair loss and a definite strengthening of the anchoring of the hair in the scalp.

The disclosure in German Patent Application 100 35 735.0 of Jul. 22, 2000 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in a composition and method for preventing or at least reducing hair loss, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A cosmetic composition for preventing or at least reducing hair loss, said composition comprising
at least one saturated or unsaturated C₁₀- to C₁₈-fatty acid, or a salt thereof; and
at least one effective ingredient selected from the group consisting of biotin and caffeine.

2. The cosmetic composition as defined in claim 1, wherein said at least one saturated or unsaturated C₁₀- to C₁₈-fatty acid is selected from the group consisting of capric acid, lauric acid, myristic acid, isomyristic acid, palmitic acid, palmitoleic acid, stearic acid, oleic acid, linoleic acid, linolenic acid, parinaric acid and petroselinic acid; and wherein said salt thereof is a physiologically acceptable salt.

3. The cosmetic composition as defined in claim 1, wherein said at least one saturated or unsaturated C₁₀- to C₁₈-fatty acid is selected from the group consisting of lauric acid and myristic acid.

4. The cosmetic composition as defined in claim 1, comprising said at least one saturated or unsaturated C₁₀- to C₁₈-fatty acid or said salt thereof and said biotin and said caffeine.

5. The cosmetic composition as defined in claim 1, further comprising at least one vasodilating substance.

6. The cosmetic composition as defined in claim 5, wherein said at least one vasodilating substance is selected from the group consisting of tocopherols, esters of tocopherols, nicotinic acid, esters of nicotinic acid, nicotinamde, camphor, Royal jelly and vasodilating plant extracts.

7. The composition as defined in claim 1, in the form of a hair tonic.

8. A composition for preventing or treating hair loss of hair on a scalp of a human being, said composition comprising
at least one solvent selected from the group consisting of water, lower univalent alcohols having one to four carbon atoms, glycerol, ethylene glycol and propylene glycol;
at least one fatty acid having from 10 to 18 carbon atoms, or a physiologically compatible salt thereof;
biotin;
caffeine;
at least one blood supply increasing substance;
at least one cosmetic additive ingredient selected from the group consisting of perfume oils, surfactants, turbidity-inducing substances, preservatives, buffer substances, hair care-providing ingredients, light-protective agents, antioxidants, radical trapping agents, anti-flaking agents, hair luster-imparting agents, vitamins, combability improving agents and defatting agents.

9. The composition as defined in claim 8, wherein said at least one blood supply increasing substance is selected from the group consisting of tocopherols, esters of tocopherols, nicotinic acid, esters of nicotinic acid, nicotinamde, camphor, Royal jelly and vasodilating plant extracts.

10. A hair tonic for preventing or treating hair loss of hair on a scalp of a human being, said hair tonic having a pH of from 4 to 6.5 and comprising
from 70 to 99.5 percent by weight of a solvent system, said solvent system comprising at least at least one solvent selected from the group consisting of water, lower univalent alcohols having one to four carbon atoms, glycerol, ethylene glycol and propylene glycol;
from 0.1 to 10 percent by weight of at least one fatty acid having from 12 to 14 carbon atoms, or a physiologically compatible salt thereof;
from 0.005 to 0.1 percent by weight of biotin;
from 0.1 to 10 percent by weight of caffeine;
at least one blood-supply-increasing substance;
at least one cosmetic additive ingredient selected from the group consisting of perfume oils, surfactants, turbidity-inducing substances, preservatives, buffer substances, hair care-providing ingredients, light-protective agents, antioxidants, radical trapping agents, anti-flaking agents, hair luster-imparting agents, vitamins, combability improving agents and defatting agents.

11. A method for prevention or treatment of hair loss, said method comprising the steps of:
a) providing an effective ingredient combination comprising at least one saturated or unsaturated C₁₀- C₁₈-fatty acid, or a salt thereof and at least one effective ingredient selected from the group consisting of biotin and caffeine; and
b) applying said effective ingredient combination to a region in which said hair loss has occurred or is possible.

12. A method of reducing hair loss and/or promoting naturally-occurring hair growth, said method comprising the steps of:
a) inhibiting the action of 5-alpha-dihydrotestosterone on head hair thickness or abundance,
b) promoting keratinization of trichocytene by introduction of regulators, and
c) increasing blood supply in the region around the hair organ;
wherein said steps are performed simultaneously by applying to the scalp in the vicinity of the hair organ an effective ingredient composition comprising at least one saturated or unsaturated C₁₀- to C₁₈-fatty acid, or a salt thereof and at least one effective ingredient selected from the group consisting of biotin and caffeine.
